(19) United States
(12) Patent Application Publication
(10) Pub. No.: US 2019/0021418 A1
(43) Pub. Date: Jan. 24, 2019

(54) PERSONAL HYGIENE KITS

(71) Applicant: Richard Jury, Colbert, WA (US)

(72) Inventor: Richard Jury, Colbert, WA (US)

(21) Appl. No.: 16/037,977

(22) Filed: Jul. 17, 2018

Related U.S. Application Data
(60) Provisional application No. 62/534,609, filed on Jul. 19, 2017.

(51) Int. Cl. A41D 19/00; A61K 8/02; A47K 10/32

(52) U.S. Cl. CPC A41D 19/002; A41D 19/01594; A47K 10/32; A61K 8/0208

(57) ABSTRACT

A glove system including a glove body and a pouch. The pouch is secured to an outside surface of the glove body and having an opening arranged therethrough. A flap is arranged with the opening of the pouch. The flap being peelable between a closed position and an open position. The flap being resealable with the opening when in the closed position.

### RELATED APPLICATIONS

This application claims the benefit of U.S. Provisional Application Ser. No. 62/534,609, filed Jul. 19, 2017, entitled “PERSONAL HYGIENE KIT,” which is hereby incorporated by reference in its entirety.

### BACKGROUND

Personal hygiene products (e.g., wet wipes, sanitizers, disposable bags, etc.) can be difficult to manage during a time of use. This is particularly true in environments having little to no facilities where the wet wipes, sanitizers, and disposable bags are strewn about and difficult to reach while attending to personal hygiene (e.g., cleaning a baby's bottom). For example, it may be difficult to position the wet wipes, sanitizers, and disposable bags proximate to the toddler, while simultaneously handling the toddler being cleaned.

Accordingly, there remains a need in the art for personal hygiene products that are easy to manage and use when desired.

### BRIEF SUMMARY

This Brief Summary is provided to introduce simplified concepts relating to glove systems and personal hygiene kits that are easy to manage and use when desired which are further described below in the Detailed Description. This summary is not intended to identify essential features of the claimed subject matter, nor is it intended for use in determining the scope of the claimed subject matter.

This disclosure relates to glove systems that are configured to contain a product for personal hygiene use. Generally, the glove systems include a pouch secured to an outside surface of a glove body for containing the product. A flap may be arranged with an opening arranged in the pouch that may peel between a closed position and an open position. The flap may be resealable with the opening when in the closed position. In some embodiments, such glove systems are easy to manage and use when desired.

In an embodiment, a glove system includes a glove body formed of a flexible material. The glove body may have an inside surface and an outside surface opposite the inside surface. A pouch including a bottom surface opposite a top surface may have the bottom surface of the pouch secured to the outside surface of the glove body. The top surface of the pouch may have an opening arranged therethrough. A cover may be arranged with the opening. The cover may be displaceable between a closed position and an open position, and the cover may be resealable with the opening when in the closed position.

In an embodiment, a glove system includes a glove body having an inside surface and an outside surface opposite the inside surface. A pouch may be secured to the outside surface of the glove body and have an opening arranged therethrough. A flap may be arranged with the opening, and the flap may be peelable between a closed position and an open position. The flap may be resealable with the opening when in the closed position.

In an embodiment, a glove system includes a first glove body formed of flexible material and a second glove body formed of the flexible material. A pouch may be secured to an outside surface of the first glove body. The pouch may have an opening arranged therethrough. A flap may be arranged with the opening and may be peelable between a closed position and an open position. The flap being resealable with the opening when in the closed position.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items.

FIG. 1 illustrates a perspective view of an example glove system including a first glove body and a second glove body.

FIG. 2 illustrates an exploded assembly view of the first glove body and a pouch.

FIG. 3 illustrates a section view of the pouch secured to the first glove body taken along section line A-A, in FIG. 1.

### DETAILED DESCRIPTION

### Overview

As noted above, personal hygiene products can be difficult to manage during a time of use, and wet wipes, sanitizers, disposable bags, etc. exist that help attend to personal hygiene, but the wet wipes, sanitizers, disposable bags, etc. can be difficult to position and may become strewn about, making the personal hygiene products difficult to reach while attending to personal hygiene. This disclosure is directed to glove systems and personal hygiene kits that are easy to manage and use when desired. The glove systems include a pouch that is secured to an outside surface of a glove body, and a flap arranged with an opening that may be peeled between a closed position and an open position. The flap may be resealable with an opening when in the closed position. The glove systems may include a product stored in the pouch. For example, the glove systems may include one or more towelettes, one or more moist towelettes, one or more cleansing pads, one or more industrial wipes, one or more pain relief pads, one or more medical wet wipes, or a combination thereof stored in the pouch. A user may peel open the flap, and remove one of the one or more towelettes, one of the one or more moist towelettes, one of the one or more cleansing pads, one of the one or more industrial wipes, one of the one or more pain relief pads, one of the one or more medical wet wipes, or a combination thereof from the pouch for personal hygiene use.

In this way, the glove systems readily position products that remain accessible for use when desired. While this application describes various embodiments of glove systems used in the field of personal hygiene, this is by way of example and not limitation. For example, the glove systems may be used in other fields such as medical applications, automotive applications, sporting applications, cosmetic applications, industrial applications, manufacturing applications etc.

The glove systems may include a glove body formed of a flexible material. The glove body having an inside surface and an outside surface opposite the inside surface. A bottom surface of a pouch may be secured to the outside surface of the glove body. The pouch may have an opening arranged therethrough. A cover may be arranged with the opening. The cover may be displaceable between a closed position and an open position, and the cover may be resealable with the opening when in the closed position.

The glove systems may include a glove body having an inside surface and an outside surface opposite the inside surface. In one example, a pouch may be secured to the outside surface of the glove body and have an opening arranged therethrough. A flap may be arranged with the opening, and the flap may be peelable between a closed position and an open position. The flap may be resealable with the opening when in the closed position.

The glove systems may include first and second glove bodies formed of a flexible material. In one example, a pouch may be secured to an outside surface of the first glove body. The pouch may have an opening arranged therethrough. A flap may be arranged with the opening and may be peelable between a closed position and an open position. The flap being resealable with the opening when in the closed position.

### Illustrative Glove Systems

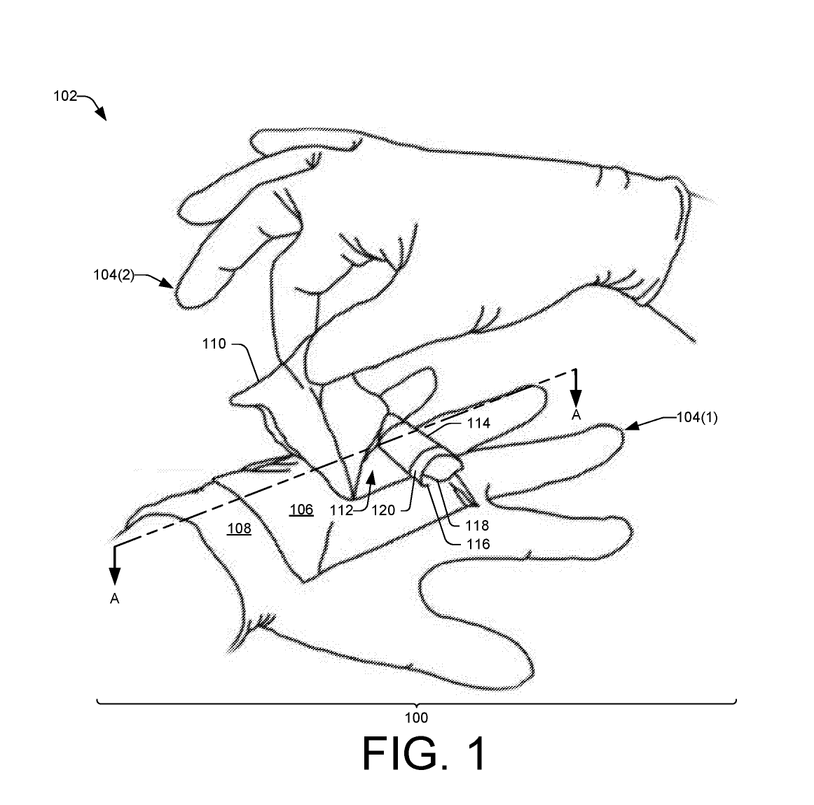

FIG. 1 illustrates a perspective view 100 of an example glove system 102 depicting cross-section line A-A. The glove system 102 may include a first glove body 104(1) and a second glove body 104(2). The first glove body 104(1) and the second glove body 104(2) may be formed of a flexible material. In one example, the flexible material may comprise a synthetic rubber (e.g., nitrile rubber). In another example, the flexible material may comprise a natural rubber (e.g., latex rubber).

A pouch 106 may be secured to an outside surface 108 of the first glove body 104(1). The pouch 106 may be formed of at least one of polyethylene (PE) or polypropylene (PP). The pouch 106 may contain a product 110. In one example, the product 110 may include one or more towelettes, one or more moist towelettes, one or more cleansing pads, one or more industrial wipes, one or more pain relief pads, one or more medical wet wipes, or a combination thereof. In another example, the product 110 may include one or more alcohol wipes, one or more petroleum jelly wipes, one or more antibiotic wipes, one or more solvent wipes, one or more cleaner wipes, one or more adhesive wipes, one or more cosmetic wipes, or a combination thereof. In another example, the product 110 may include one or more glove bodies (e.g., one or more first or second glove bodies 104(1) or 104(2)).

The pouch 106 may have an opening 112 arranged therethrough. The opening 112 may provide for removing the product 110 from the pouch 106. For example, the opening 112 may provide for removing one of the one or more towelettes, one of the one or more moist towelettes, one of the one or more cleansing pads, one of the one or more industrial wipes, one of the one or more pain relief pads, one of the one or more medical wet wipes, or a combination thereof from the pouch 106 for use.

A cover 114 may be arranged with the opening 112. For example, the cover 114 may be a flap having an end 116 that is attached to the pouch 106 and an end 118 that is free and not attached to the pouch 106. The cover 114 being displaceable between a closed position and an open position. For example, the cover 114 may be peelable between the closed position and the open position. When in the closed position, the cover 114 is disposed over the opening 112, and when in the open position the cover 114 is disposed away from the opening 112. The cover 114 may be resealable with the opening 112 when in the closed position. For example, the cover 114 may include an adhesive 120 (e.g., a pressure-sensitive adhesive, self-adhesive, self-stick adhesive, etc.) arranged along a perimeter of the cover 114 that adheres to the pouch 106 when the cover 114 is in the closed position.

While FIG. 1 illustrates the cover 114 is a flap that is peelable between the closed position and the open position, the cover 114 may be a rigid (e.g., inflexible, stiff, firm, etc.) cap that is pivotable between the closed position and the open position. For example, the cover 114 may be a rigid cap attached to the pouch 106 via a hinge (e.g., a flexure bearing, a living hinge, etc.) that provides for pivoting the cap between the closed position and the open position.

Further, while FIG. 1 illustrates the glove system 102 includes first and second glove bodies 104(1) and 104(2), the glove system 102 may include any number of glove bodies. In one example, the glove system 102 may include one glove body having the pouch 106 attached to the single glove body. In another example, the glove system 102 may not include a glove body. For example, the glove system 102 may include a pouch (e.g., pouch 106) having an adhesive (e.g., a pressure-sensitive adhesive, self-adhesive, self-stick adhesive, etc.) disposed on the bottom surface of the pouch that provides for a user to adhere the pouch to a glove body. Still further, while FIG. 1 illustrates the glove system 102 includes one pouch (e.g., pouch 106) secured to the first glove body 104(1), the glove system 102 may further include a second pouch secured to the second glove body 104(2). The pouch 106 secured to the first glove body 104(1) may contain a first product (e.g., product 110), and the second pouch secured to the second glove body 104(2) may contain a second product different than the first product.

While FIG. 1 illustrates the first and second glove bodies 104(1) and 104(2) being zero porosity single use medical exam gloves, the first glove body 104(1) and/or the second glove body 104(2) may be other types of gloves. For example, the first glove body 104(1) and/or the second glove body 104(2) may be household rubber latex cleaning gloves, rubber mechanic gloves, rubber gardening gloves, etc.

The glove system 102 may be a personal hygiene kit. The personal hygiene kit may further include one or more of the following items:
one 2-4 mil thick re-sealable zip-lock style plastic bag;
a changing pad that may be training pant style;
an absorbent undergarment sized for toddlers or small children; and/or
a 6.00 mil thick double sealed, opaque plastic bag for temporary smell resistant storage of fecal contaminated clothing and under garments.

At a time of a personal hygiene event (e.g., a cleaning of a bottom of a toddler) and/or in the absence of sanitation facilities, a user may don both of the first and second glove bodies 104(1) and 104(2). The user may put the first glove body 104(1) on a non-dominant hand of the user, such that the pouch 106 containing the product 110 (e.g., moist towelettes) is arranged on a back of the non-dominant hand. The user may put the second glove body 104(2) on the dominant had of the user. The user may separate contaminated clothing from the toddler. Using the non-dominant hand, the user may hold the feet and/or the ankles of the toddler to (1) prevent the feet from kicking and possibly getting into the contamination, and (2) to keep the area (e.g., the bottom of the toddler) needing to be wiped steady for ease of cleanup. Using the dominant hand, the user may peel the cover 114 to the open position and begin drawing out (e.g., removing) up to five moist towelettes, one moist towelette of the five moist towelettes at a time as needed while holding the feet and/or ankles with the non-dominant hand. Because the pouch 106 containing the moist towelettes is positioned directly in front of the user and on the back of the non-dominant hand of the user, the user can easily manage and use the moist towelettes when desired. The user may store contaminated towelettes, contaminated clothing and/or contaminated under garments in the 6.00 mil thick double sealed, opaque plastic bag that provides for little to no odor until proper disposal is obtained. Further, the user may store contaminated towelettes, contaminated clothing and/or contaminated under garments in the 6.00 mil thick double sealed, opaque plastic bag that further provides for preventing visibility of the contaminated towelettes, contaminated clothing and/or contaminated under garments until proper disposal is obtained. Thus, subsequent to the end of the personal hygiene event, the user may engage in another activity (e.g., cooking, cleaning, working, etc.) with the confidence and surety, knowing that the user's hands are just as clean as before the personal hygiene event.

The glove system 102 may utilize a color coding system. For example, the glove system 102 may utilize a color coding system so a user may easily identify the glove system 102. For that a red glove body and/or pouch may indicate that a dangerous product (e.g., dangerous solvent wipe) may be contained in the pouch. In another example, a blue glove body and/or pouch may indicate that a child safe product (e.g., child safe cleaning wipe) may be contained in the pouch. The glove system 102 may provide for germproof, compact, easily stowable, and environmentally friendly personal hygiene care. The glove system 102 may provide for a single-use application.

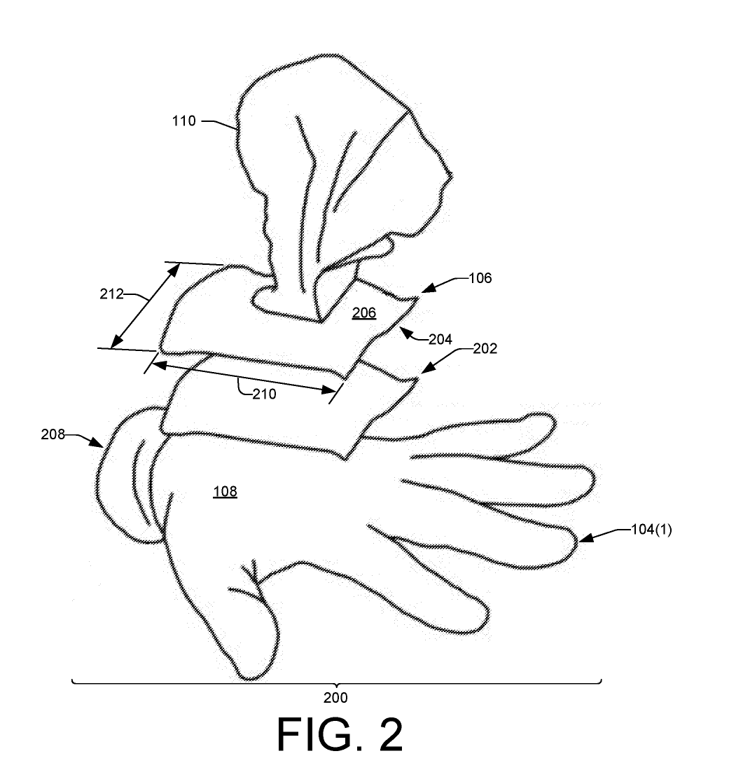

FIG. 2 illustrates an exploded assembly view 200 of the first glove body 104(1) and the pouch 106. The glove system 102 may further include an adhesive patch 202. The adhesive patch 202 may have a length of about 3.75 inches and a width of about 2 inches. The adhesive patch 202 may be a high-strength bonding tape. The pouch 106 including a bottom surface 204 opposite a top surface 206. The bottom surface 204 of the pouch 106 for securing the pouch 106 to the outside surface 108 of the first glove body 104(1) opposite an inside surface 208 of the first glove body 104(1). The adhesive patch 202 is disposed such that the adhesive patch 202 secures the bottom surface 204 of the pouch 106 to the outside surface 108 of the first glove body 104(1).

While FIG. 2 illustrates the pouch 106 secured to the outside surface 108 of the first glove body 104(1) via the adhesive patch 202, the pouch 106 may be secured to the outside surface 108 of the first glove body 104(1) via a spray on adhesive (e.g., a spray on contact cement), a mechanical fastener (e.g., threaded fastener), an interference fit, a press fit, a friction fit, a magnet, a suction cup, a hook and loop fastener, a clip, a cam lock, etc. Further, while FIG. 2 illustrates the pouch 106 secured to the outside surface 108 of the first glove body 104(1), the pouch 106 may be formed integrally with the first glove body 104(1). For example, the pouch 106 and the first glove body 104(1) may be formed (e.g., molded, injection molded, cast, etc.) of one single unit of flexible material (e.g., synthetic rubber, natural rubber, etc.). In another example, the pouch 106 may be over-molded onto the first glove body 104(1).

The pouch 106 may have a length 210 of about 3.75 inches and a width 212 of about 2.4 inches. The pouch 106 may have a size suitable to contain a quantity of about five pieces of the product 110. For example, the pouch 106 may have a size suitable to contain about five towelettes. Each of the five towelettes may have a length of about 6 inches and a width of about 4 inches.

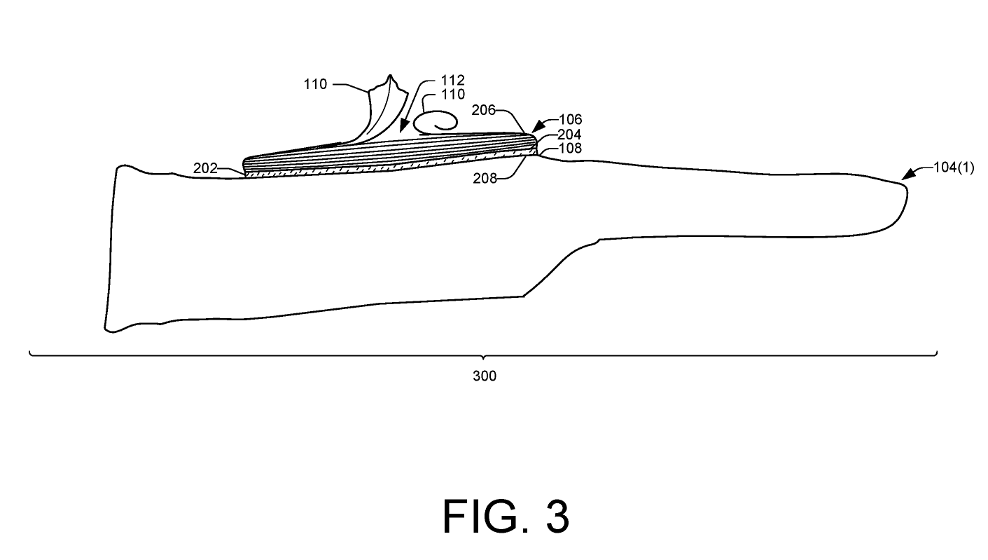

FIG. 3 illustrates a section view 300 of the pouch 106 secured to the first glove body 104(1) taken along line A-A, in FIG. 1. The section view 300 illustrates the bottom surface 204 of the pouch 106 may be secured to the outside surface 108 of the first glove body 104(1) via the adhesive patch 202. For example, the adhesive patch 202 may be sandwiched between the bottom surface 204 of the pouch 106 and the outside surface 108 of the first glove body 104(1).

The outside surface 108 of the first glove body 104(1) is opposite to the inside surface 208 of the first glove body 104(1). A hand of the user may interface with the inside surface 208 of the first glove body 104(1) when the user wears the first glove body 104(1). The bottom surface 204 of the pouch 106 is opposite to the top surface 206 of the pouch 106. The product 110 may be removed from the opening 112 arranged in the top surface 206 of the pouch 106 when the cover 110 is peeled to the open position.

### CONCLUSION

Although the invention has been described in language specific to structural features and/or methodological acts, it is to be understood that the invention is not necessarily limited to the specific features or acts described. Rather, the specific features and acts are disclosed as illustrative forms of implementing the invention. For example, while embodiments are described having certain shapes, sizes, and configurations, these shapes, sizes, and configurations are merely illustrative.

What is claimed is:

1. A glove system comprising:
a glove body formed of a flexible material, the glove body having an inside surface and an outside surface opposite the inside surface;
a pouch including a bottom surface opposite a top surface, the bottom surface secured to the outside surface of the glove body, and the top surface having an opening arranged therethrough; and
a cover arranged with the opening, the cover being displaceable between a closed position and an open position, and the cover being resealable with the opening when in the closed position.

2. The glove system of claim 1, wherein the pouch contains a product.

3. The glove system of claim 2, wherein the product includes one or more towelettes, one or more moist towelettes, one or more cleansing pads, one or more industrial wipes, one or more pain relief pads, one or more medical wet wipes, or a combination thereof.

4. The glove system of claim 1, wherein the flexible material comprises a synthetic rubber.

5. The glove system of claim 4, wherein the synthetic rubber is nitrile rubber.

6. The glove system of claim 1, further comprising an adhesive patch, and wherein the adhesive patch is disposed such that the adhesive patch secures the bottom surface of the pouch to the outside surface of the glove body.

7. The glove system of claim 1, wherein the pouch is formed of at least one of polyethylene (PE) or polypropylene (PP).

8. The glove system of claim 1, wherein the pouch has a length of about 3.75 inches and a width of about 2.4 inches.

9. The glove system of claim 8, wherein the pouch has a size suitable to contain about five towelettes, each of the five towelettes having a length of about 6 inches and a width about 4 inches.

10. A glove comprising:
a glove body having an inside surface and an outside surface opposite the inside surface;
a pouch secured to the outside surface of the glove body, the pouch having an opening arranged therethrough; and
a flap arranged with the opening, the flap being peelable between a closed position and an open position, and the flap being resealable with the opening when in the closed position.

11. The glove system of claim 10, wherein the glove body comprises a synthetic rubber.

12. The glove system of claim 10, further including an adhesive patch, and wherein the adhesive patch secures the pouch to the glove body.

13. The glove system of claim 10, wherein the pouch is formed of at least one of polyethylene (PE) or polypropylene (PP).

14. The glove system of claim 10, wherein the pouch has a length of about 3.75 inches and a width of about 2.4 inches.

15. The glove system of claim 10, wherein the pouch has a size suitable to contain about five towelettes, each of the five towelettes having a length of about 6 inches and a width about 4 inches.

16. The glove system of claim 10, wherein the pouch contains one or more glove bodies.

17. A glove system comprising:
a first glove body formed of flexible material;
a second glove body formed of the flexible material;
a pouch secured to an outside surface of the first glove body, the pouch having an opening arranged therethrough; and
a flap arranged with the opening, the flap being peelable between a closed position and an open position, and the flap being resealable with the opening when in the closed position.

18. The glove system of claim 17, further including an adhesive patch, and wherein the adhesive patch secures the pouch to the first glove body.

19. The glove system of claim 17, wherein the pouch has a size suitable to contain about five towelettes, each of the five towelettes having a length of about 6 inches and a width about 4 inches.

20. The glove system of claim 17, wherein the flexible material is a synthetic flexible rubber, and the pouch is formed of at least one of polyethylene (PE) or polypropylene (PP).
